(19) United States
(12) Patent Application Publication
(10) Pub. No.: US 2024/0024279 A1
(43) Pub. Date: Jan. 25, 2024

(54) METHODS FOR REDUCING THE WEIGHT LOSS OR INCREASING THE WEIGHT OF A FELINE IN NEED THEREOF

(71) Applicant: INCREVET, INC., Boston, MA (US)

(72) Inventors: Michael HADD, San Jose, CA (US); Brian SEED, Boston, MA (US)

(21) Appl. No.: 18/131,727

(22) Filed: Apr. 6, 2023

Related U.S. Application Data
(63) Continuation of application No. 16/856,342, filed on Apr. 23, 2020, now abandoned.
(60) Provisional application No. 62/839,248, filed on Apr. 26, 2019.

(51) Int. Cl. A61K 31/351; A61P 3/10

(52) U.S. Cl. CPC A61K 31/351; A61P 3/10; A61K 9/0053

(57) ABSTRACT

Provided herein are methods of reducing the weight loss and/or increasing the weight of a feline in need thereof, said methods include administering to a feline in need thereof a total daily dosage of about 2 to 50 mg of Compound 1, having the formula:

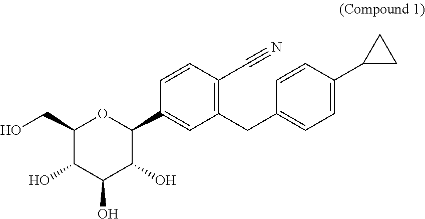 [SMILES: N#Cc1ccc([C@@H]2O[C@H](CO)[C@@H](O)[C@H](O)[C@H]2O)cc1Cc1ccc(C2CC2)cc1]

or a pharmaceutically acceptable form thereof. Also provided herein are methods of managing the disease of a feline with diabetes mellitus and elevated IGF-1 concentration.

### CROSS-REFERENCES TO RELATED APPLICATIONS

This application is a continuation of U.S. application Ser. No. 16/856,342, filed Apr. 23, 2020, which claims priority to U.S. Provisional Application No. 62/839,248, filed Apr. 26, 2019, which is incorporated by reference in its entirety and for all purposes.

### STATEMENT AS TO RIGHTS TO INVENTIONS MADE UNDER FEDERALLY SPONSORED RESEARCH AND DEVELOPMENT

Not applicable

### REFERENCE TO A “SEQUENCE LISTING,” A TABLE, OR A COMPUTER PROGRAM LISTING APPENDIX SUBMITTED ON A COMPACT DISK

Not applicable

### BACKGROUND OF THE INVENTION

Diabetes is a disease characterized by a sustained high blood glucose concentration (hyperglycemia) that adversely affects multiple organ systems and in severe cases can result in death. Diabetes has been observed in companion animals such as cats, dogs and horses as well as humans. As in human populations, diabetes in cats is an increasing health problem, and is associated with both advancing age and obesity.

In veterinary medicine, the objectives of a therapy take into consideration the perceptions and needs of the owner or person responsible for the care of an animal. A strategy for disease treatment that increases the objective health of the animal but that has adverse consequences on the owner, for example to increase owner anxiety over the health of the animal, would be considered inferior to a strategy that has less objective efficacy but decreases the owner's anxiety. As a result, veterinary objectives emphasize disease management, distinguished from simple disease treatment.

There are two major types of diabetes mellitus in humans: Type 1 (T1DM), also known as insulin dependent diabetes (IDDM) and Type 2 (T2DM), also known as insulin independent or non-insulin dependent diabetes (NIDDM).

Type 1 diabetes results from a failure of the body to produce insulin, most often due to a loss of the endocrine cells known as β cells, which in many organisms are found in clusters, called islets, within the exocrine pancreas. Type 2 diabetes is a disease of insulin resistance, in which the islet β cells are capable of producing insulin, but the tissues responsible for taking up glucose from the blood do not respond adequately to the insulin that is produced. Over the course of sustained T2DM, β cells may become less capable of producing insulin, and/or may succumb to the stress of constant high insulin production. In such a setting exogenous insulin may be required to maintain normal glucose levels (euglycemia or normoglycemia). However, a risk of administration of too much insulin is hypoglycemia, which can result in coma and death.

Insulin lowers the concentration of glucose in the blood by stimulating the uptake and metabolism of glucose by liver, muscle and adipose tissue. Insulin stimulates the storage of glucose in the liver and muscles as glycogen, and in adipose tissue as triglycerides. Insulin also promotes the utilization of glucose in muscle for energy. Thus, insufficient insulin levels in the blood, or decreased sensitivity to insulin, gives rise to excessively high levels of glucose in the blood. When the glucose concentration in the blood rises above a certain critical level, called the renal threshold for glucosuria, glucose begins to appear in the urine. Prior to the advent of reliable blood-based tests for diabetes, the appearance of glucose in urine was often the first indication of the disease.

Under normal conditions the kidneys allow low molecular weight compounds, including glucose, in the blood to exit the body in the glomerular filtrate. From the filtrate, the kidneys selectively recover nearly all of the water, sodium, potassium and chloride ions, as well as important metabolites including vitamins, glucose and other sugars, and amino acids. The energetically expensive process of discarding nearly the entire contents of plasma, then selectively reabsorbing only those components of interest, underlies the ability of the kidney to discharge toxins of great and unpredictable diversity. The mechanism for reabsorption of glucose also provides a homeostatic capability when plasma glucose levels rise too high. When the concentration of glucose in the blood exceeds the renal threshold for glucosuria, some of the excess glucose is discharged in the urine, palliating to some extent the adverse consequences of elevated plasma glucose.

As the degree of glucosuria rises, an increase in urinary output is observed, the consequence of a phenomenon known as osmotic diuresis. Glucose in the filtrate osmotically prevents the concentration of urine by water reabsorption, leading to a higher rate of loss of water, in turn resulting in dehydration. Dehydration causes increased thirst and water consumption. The inability to utilize glucose energy eventually leads to weight loss despite an increase in appetite. Excessive water consumption (polydipsia), food consumption (polyphagia or hyperphagia) and urine production (polyuria) are common symptoms of advanced diabetes.

The toxic effects of excess plasma levels of glucose include the non-enzymatic (spontaneous) glycosylation of cells and tissues. Glycosylated products accumulate in tissues and may eventually form cross-linked proteins, which are termed advanced glycation end products. Although the detailed mechanisms are for the most part unknown, it is well understood that diabetes elevates the likelihood or severity of several conditions and can result in painful neuropathies, impaired circulation, gangrene, amputation, renal failure, blindness, myocardial infarction and stroke.

The determination of the degree of overall glycemic control is an important element of a diabetes management plan. Measurement of serum glucose by an analytical laboratory, or of blood glucose with a point-of-care device such as a home glucometer, can aid in assessing glycemic status. Glucometers measure glucose in whole blood, whereas analytical laboratories typically measure glucose in serum, the liquid phase resulting from blood coagulation. Individual serum or blood glucose values can vary substantially throughout the day, typically rising after meals and falling after prolonged fasting. Such daily fluctuations can reduce the utility of serum or blood sampling for determining the degree of glycemic control resulting from a given therapeutic intervention.

To provide a better assessment of the degree of control of feline diabetes, it is common to take multiple blood samples over the course of a day, typically as an inpatient study in which the cat is housed in a veterinary clinic for the duration of the measurements. The resulting profiles of blood glucose as a function of time are known as blood glucose curves, and are often conducted to confirm an initial diagnosis, or assess the effectiveness of a management plan.

Confinement of a cat in a veterinary clinic can be stressful to a cat, and one of the documented consequences of stress is hyperglycemia. Hence blood glucose curves obtained in a clinic, although rarely confounded by technical limitations affecting the blood draw or measurement accuracy, can consist of unreliably high values that reflect the action of cortisol, the principal hormone released in stress, and not the natural course of fluctuation of the cat's daily blood glucose concentration. Hence measurements in the clinic always need to be evaluated for their potential to underestimate the degree of glycemic control.

According to guidelines published by the International Society for Feline Medicine (Sparkes et al., 2015; J Feline Med Surg 17:235), the primary goal of management of feline diabetes, as measured by blood glucose curve criteria, is to maintain the blood glucose between a peak of 10-14 mmol/L (180-252 mg dL⁻¹) and a nadir of 4.5-8.0 mmol/L (80-144 mg dL⁻¹). Both peak and nadir are specified because the only approved medication for diabetes is insulin, which if administered in excess can cause a dangerous hypoglycemia. According to this guideline, a cat with diabetes is adequately managed if its blood glucose curve measurements fall between 80 and 252 mg dL⁻¹.

Related guidelines published by the American Animal Hospital Association (Rucinsky et al., 2010 J Am Anim Hosp Assoc 46:215) recommend the conduct of an at-home blood glucose curve having a target for average blood glucose of less than 250 mg dL⁻¹ with no single blood glucose measurement greater than 300 mg dL⁻¹ and a nadir of 80-150 mg dL⁻¹.

Blood glucose curves are inconvenient and expensive to conduct. As an alternative, surrogate measures of glycemic control that reflect the average blood glucose concentration over a long period of time, such as weeks to months, and that can be measured in a single blood sample, can be deployed.

In humans non-enzymatically glycated hemoglobin provides a convenient method to determine long term glycemic control. The N-terminal valine residues of hemoglobin A1 undergo a spontaneous chemical reaction with reducing sugars, of which glucose is the most abundant in the blood. The initial step is the formation of an enamine (Schiff base) between the glucose aldehyde tautomer and the N-terminal amino group. The second step, called an Amadori rearrangement, results in a tautomerization to produce a β-keto amine, often referred to as an Amadori adduct. Because the average lifetime of a human red blood cell is about 120 days, the degree of non-enzymatic glucosylation represents the average accumulation of glycation products over a mean period of half this time. Measurement of the fraction of glycated hemoglobin is the basis of an assay referred to as the hemoglobulin A_{1c} (HbA_{1c}) assay. A sample of blood in which the percentage of HbA_{1c} is less than 6.5 is typically considered to reflect good or adequate glycemic control, whereas higher percentages are typically interpreted as indicating the presence of diabetes.

The erythrocytes of cats have shorter half-lives than human erythrocytes, and the HbA_{1c} levels are much lower, and less precisely measured. Instead, the preferred measure of sustained glycemic status in cats is the serum fructosamine assay, which also measures the non-enzymatic adducts created by the reaction of reducing sugars with primary amines. Mechanistically, these adducts are formed by the same reaction sequence as with the N-terminal amino group of hemoglobin, but can form on the ϵ-amino side chains of lysine, forming, in the case of glucose, a structure known as fructoselysine. The fructosamine test measures total serum keto amines by reversing the Amadori rearrangement. Under alkaline conditions, the Amadori products revert to the original enamines, which reduce nitroblue tetrazolium to a colored formazan dye that is quantitated spectrophotometrically at 540 nm. The fructosamine assay measures total β-keto amines, but the largest component is that attributable to serum albumin, the most abundant protein in plasma. Albumin has a half-life of approximately 20 days, so the fructosamine measurement effectively measures the three-week history of glycemic control.

According to guidelines published by the International Society for Feline Medicine (Sparkes et al., 2015; J Feline Med Surg 17:235), a serum fructosamine level of less than 350 μmol L⁻¹ indicates either excellent glycemic control, insulin overdose or diabetic remission; of 350-450 μmol L⁻¹ indicates good glycemic control; of 450-550 μmol L⁻¹ indicates moderate glycemic control; and of >550 μmol L⁻¹ indicates poor glycemic control. These value ranges are predicated on testing laboratory methodology that places the upper limit of normal for serum fructosamine at approximately 350 μmol L⁻¹.

At present there are no approved oral hypoglycemic agents for the management of diabetes in cats. The standard of care for feline diabetes requires twice daily injections of insulin, titrated to desired effect. Cats show substantial inter-individual variation in insulin sensitivity and must be carefully observed to ensure that a fatal or neurologically catastrophic hypoglycemia does not occur. Although the administration of insulin can help control diabetes and slow disease progression, providing the proper dose and timing of the insulin can be challenging. For example, it is recommended that the administration of insulin be timed around a meal, but consistent timing of insulin with meals can be difficult to arrange and often results in a lower level of compliance.

As such, there is a need in the art for improved methods for reducing the hyperglycemia of feline diabetes and the hyperglycemia-associated clinical signs, and particularly for methods that do not entail injections and that do not require careful dose adjustment to preserve the wellbeing of the cat. The present disclosure addresses this need and provides related advantages. In particular the disclosure provides methods for the management of feline diabetes that rely on administration of velagliflozin.

Much of what is known about these proteins comes from studies of rodents and humans and is not necessarily applicable to cats. Cats are obligate carnivores and ordinarily consume very little carbohydrate. Cats lack receptors for sweet taste that are present in rodents and humans and the receptors and transporters responsible for carbohydrate sensing and movement cannot be assumed to function in the same manner as their cognates in rodents or humans. What follows, therefore, is an accounting of the general properties of carbohydrate transport as reflected by the largest body of current understanding, which may differ in material ways from the description of transport in cats.

Because glucose does not spontaneously diffuse across cell membranes, rodents and humans have two classes of integral membrane protein, called transporters, to facilitate the movement of glucose from the extracellular medium into the cell. One class, called ‘equilibrative’, does not favor the interior or the exterior, but rather allows glucose to move from the region of higher concentration to the region of lower (moving in the direction of equilibrium). Since cells consume glucose, this results in a net flux into the cell in most cases. The other class, called ‘concentrative’, relies on the natural gradient of sodium ions from the extracellular to the intracellular compartments. The gradient is sustained by active pumping of Na⁺ by an energy-requiring (ATP-consuming) mechanism that exchanges intracellular Na⁺ for extracellular K⁺, thereby increasing the extracellular concentration of Na⁺ and the intracellular concentration of K⁺. Sodium-glucose linked transporters (SGLTs) transport one glucose and one Na⁺ ion (in the case of SGLT2) or one glucose and two Na⁺ ions (in the case of SGLT1) across the membrane in a single action. The Na⁺ ions effectively carry the glucose with them into the cell. Concentrative transporters thus allow dilute extracellular glucose to be concentrated in the cell interior.

Most cells exhibit only equilibrative transport. The intestines and kidneys rely on concentrative transport to take up glucose from the diet or to retrieve glucose from urine. In the species studied to date SGLT1 is present in both intestine and kidney, whereas SGLT2 is found in the kidney, anatomically upstream of SGLT1 in the renal tubules. SGLT2 acquires glucose at a lower Na⁺ cost than SGLT1, and under normal conditions is responsible for the reuptake of approximately 90% of the glucose in filtrate. In the absence of SGLT2, SGLT1 partially compensates, and 40-50% of the glucose is retained. The rest is lost to urine. Genetic deficiencies of SGLT2 are known in mice and humans and are generally benign, occult syndromes detected in humans only by random urine testing. Genetic deficiency of SGLT1 is a potentially lethal condition in humans, due to a severe diarrhea that can only be managed by strict dietary limitation of carbohydrate. Both SGLT1 and 2 cotransport large amounts of water with Na⁺ and glucose. In humans and rodents, SGLT1 can transport both glucose and galactose. Whether galactose is a substrate for feline SGLT1 has yet to be reported.

Much of the early work on the physiology of renal reuptake of glucose was facilitated by the identification of a natural product, phlorizin, isolated from the bark of apple trees, that was eventually found to be an inhibitor of SGLT1 and SGLT2 in multiple species.

Phlorizin (also known in the literature as phloridzin, phloridizin, phlorhizin and phloridzine) was noted in the 19^{th} century to promote glucosuria. At the time, because diabetes was characterized chiefly by the presence of glucose in urine, phlorizin was considered to be diabetogenic, and the early literature refers to “phlorizin diabetes.” It was quickly recognized, however, that the glucosuria elicited by phlorizin resulted from a different mechanism than the glucosuria resulting from pancreatic damage or removal, and before the turn of the century, E. Hédon (Compt Rend Soc Biol 4:60 1897) reported the correction of experimental diabetes in dogs by the administration of phlorizin. A translation of his observations from French could be made as follows: “Another fact that, to my knowledge, has not been observed yet, is that when phloridzine is administered to pancreatectomized animals in full glycosuria, the hyperglycemia disappears; we see then an inverse relation between glycosuria and glucose; while the former is growing in a large proportion (as Minkowski has seen), the second decreases until it returns to the normal state.” By the 1920s the action of phlorizin on the kidney had been elucidated and a comprehensive review of its actions had been published (Nash Physiol. Rev. 7: 385 1927).

A report of the use of phlorizin to reverse the hyperglycemia produced by experimental diabetes in cats was reported in 1943 by Lukens and coworkers (Lukens et al., Endocrinology 32:475 1943), and followed up by additional observations published in 1961 (Lukens et al., Diabetes 10:182 1961). The salutary effects of phlorizin on diabetes in cats included relief of stress on the pancreatic insulin-producing islet cells as well as a dramatic reduction of hyperglycemia. Lukens et al. demonstrated many effects that are considered characteristic of the action of a hypoglycemic agent on diabetic cats. They showed, for example, that phlorizin could protect animals from the development of diabetes, could restore normal glucose tolerance to diabetic animals, and could prevent exhaustion of the islets of Langerhans in the presence of experimental diabetes (Lukens et al., 1943, 1961). A key element of the studies made by Lukens and coworkers was the administration of phlorizin by subcutaneous injection of a suspension of the compound in olive oil. This effectively served as a sustained release depot formulation that delivered the active agent over a period of days. Without this strategy it is likely that their results could not have been attained. An 0-glucoside, phlorizin is susceptible to the metabolic action of β-glucosidases and has a short half-life in most species.

Recently US 2015/0164856A1 has taught the use of one or more SGLT2 inhibitors to treat diabetes in feline animals. The publication teaches the uses of SGLT2 inhibitors are various, and can, for example, prevent the loss of pancreatic beta cell mass or prevent beta cell degeneration, prevent or treat diabetes, and treat a wide variety of diabetes-related ailments or conditions. US 2015/0164856A1 can be distinguished from the very similar demonstrations and assertions of Lukens and coworkers by the emphasis on SGLT2 inhibition. Phlorizin, as a mixed SGLT1/2 inhibitor in most species, has effects on two transporters, whereas US 2015/0164856A1 teaches inhibition solely of SGLT2. Indeed, US 2015/0164856A1 makes no mention of SGLT1. It is also worth noting that the selectivity of phlorizin for feline SGLT1 and SGLT2 is not presently known, nor is the relative contribution of the two transporters to renal reuptake of glucose in the cat.

US 2015/0164856A1 also does not disclose that, as is well known in the art, prediction of the actions of a compound in one species based on experience in another is one of the most hazardous prognostications in drug development. Due to evolutionary variation in the structure of target proteins, it is quite difficult to predict actions across species with any precision. Moreover species-dependent off-target effects are well known in the art. The rate and type of metabolism of xenobiotics are important determinants of drug exposure, and are known to exhibit great variability from one species to another. In part, this is likely due to the strong genetic selection that is applied to xenobiotic metabolism pathways, which allow some species to consume food sources that are otherwise toxic to other species.

US 2015/0164856A1 teaches that velagliflozin can be used to reduce the weight of a cat, for example at paragraph 0037: “The invention is also associated with anti-obesity effects, and may in particular advantageously prevent weight gain and/or lead to a decrease in body mass in a feline animal. In one aspect, the invention thus allows obesity and/or obesity-related metabolic disorders to be managed in a feline animal.” US 2015/0164856A1 also claims the use of velagliflozin to treat obesity, among other conditions. US 2015/0164856A1 identifies secondary diabetes mellitus as one of three principal forms of diabetes mellitus, but does not teach that velagliflozin can be used to manage secondary diabetes mellitus.

### BRIEF SUMMARY OF THE INVENTION

Provided herein are methods (i) of increasing the weight of cats, (ii) of reducing the weight loss of cats, and (iii) of managing the diabetes of cats with diabetes and elevated IGF-1 levels, said methods include administering to a feline in need thereof a total daily dosage of about 2 to 50 mg of Compound 1, (velagliflozin, CAS Number 946525-65-1), having the formula:

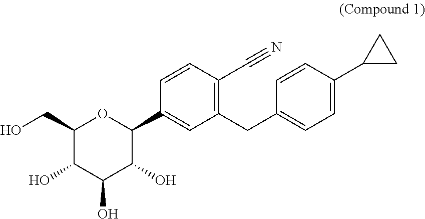 [SMILES: N#Cc1ccc([C@@H]2O[C@H](CO)[C@@H](O)[C@H](O)[C@H]2O)cc1Cc1ccc(C2CC2)cc1]

or a pharmaceutically acceptable form thereof.

### BRIEF DESCRIPTION OF THE DRAWINGS

Not applicable

### DETAILED DESCRIPTION OF THE INVENTION

### I. General

The present invention discloses surprising new uses for velagliflozin. For example, the current disclosure describes the surprising reduction in weight loss and increase in the weight of felines produced by velagliflozin, as well as the surprisingly effective control by velagliflozin of diabetes in felines with diabetes and elevated plasma IGF-1 concentration.

Velagliflozin is a C-aryl glucoside that has been studied for potential management of diabetes in non-human species. Recently Hoenig 2017 reported the action of velagliflozin in obese cats (thought to be at risk for the development of diabetes). The experimental cohorts consisted of three male and three female cats, assigned to receive either placebo or velagliflozin. The velagliflozin dosage was 1 mg/kg, administered in gelatin capsules. The placebo article was an empty gelatin capsule. Table 1 of Hoenig 2017 shows that over the course of the treatment, cats in the placebo group increased in weight from 6.6±0.9 kg to 6.7±0.8 kg, whereas cats in the velagliflozin group decreased in weight from 7.2±1.5 kg to 7.0±1.4 kg. The food intake of cats in the placebo group decreased from 62±12 g to 60±10 g whereas in the velagliflozin group it increased from 57±8 g to 58±11 g. Increases in food consumption and decreases in weight, are expected physiological consequences of velagliflozin administration. For weight gain and food consumption, the effects observed in the placebo group were opposite to those seen in the velagliflozin group.

This application teaches that a surprising and unexpected therapeutic benefit is the gain in weight of treated cats, an effect that runs counter to the expected action of the compound in both healthy and diabetic animals. Weight loss in cats administered an SGLT2 inhibitor is taught by US 2015/0164856.

As is apparent to those of skill in the art, the effectiveness of a medication is dependent on many factors, including the severity and duration of disease, the rate of metabolism of the active ingredient or its active metabolites, and the regularity with which the medication is administered. Errors in dosing, particularly omissions, can have a significant impact on the apparent utility of a medication. In actual practice, omissions are common and the degree to which omissions occur can be a determining factor in the effectiveness of a medication.

Comorbidities may also affect the effectiveness of a medication. For example, because SGLT inhibitors block renal reuptake of glucose, they can be expected to lose potency as renal filtration declines, either as a natural consequence of aging or because of advancing renal disease.

When humans initially present with T2DM, they are rarely in the throes of a diabetic crisis. Instead, the disease gradually evolves and the diagnosis is made incidental to results from regular examinations, or because of patient complaints, such as thirst or frequent urination, that herald more advanced disease. However, cats with diabetes are often presented to the veterinarian in acute disease, with very high blood glucose levels, glucosuria, and weight loss. Characteristic hyperglycemia-associated clinical signs in cats include polydipsia (excessive water consumption), polyphagia (excessive food consumption), polyuria (excessive urination) and weight loss. Often it is the weight loss and malaise that trigger the owner's concern. Surprisingly, although cats with diabetes are undernourished because of the massive loss of glucose in their urine (by the renal mechanisms detailed above), administration of velagliflozin, which increases urinary glucose secretion, paradoxically arrests the weight loss and allows many of the cats to gain weight.

A definitive explanation for the mechanism of prevention of weight loss by velagliflozin in diabetic cats has yet to be presented. However, it is possible that, by inhibiting glucose reuptake enough additional glucose is discharged in the urine to result in a reduction in plasma glucose concentration. As the plasma glucose concentration drops, the proportion of glucosuria that is due to the effects of medication, as opposed to disease, increases, further improving the glycemic control. Eventually the rate of glucose excretion balances the excess glucose production and the weight increases.

Diabetes in cats may be associated with acromegaly, a condition resulting from hyperplasia of the somatotrope compartment of the pituitary gland and resulting in inappropriate production of growth hormone. Elevated growth hormone in turn produces elevated insulin-like growth factor 1 (IGF-1), which antagonizes the action of insulin. Insulin resistance in diabetic cats is frequently associated with acromegaly (Scott-Moncrieff, J C, Vet Clin North Am Small Anim Pract (2010) 40:241), and it has been estimated that up to a quarter of diabetic cats in Europe may have underlying acromegaly (Niessen PLoS One. 2015 10:e0127794). Often very high doses of insulin are required to manage diabetic cats with acromegaly. An effective insulin-independent anti-diabetic agent would be an important addition to the options for managing feline diabetes.

Described herein are methods for reducing weight loss in cats with diabetes by administering Compound 1 (velagliflozin). The methods described herein include specific dosing amounts and frequencies.

### I. Definitions

“Compound 1” refers to the chemical 2-(4-cyclopropylbenzyl)-44(2S,3R,4R,5S,6R)-3,4,5-trihydroxy-6-(hydroxymethyl)tetrahydro-2H-pyran-2-yl)benzonitrile, having the formula:

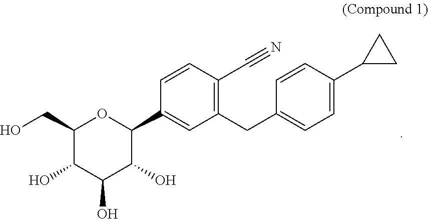 [SMILES: N#Cc1ccc([C@@H]2O[C@H](CO)[C@@H](O)[C@H](O)[C@H]2O)cc1Cc1ccc(C2CC2)cc1]

As used herein, “clinical remission” refers to a sustained reduction, abatement, or lessening in one or more clinical measures for a disease that cause the measures to fall within the accepted bounds for test values obtained from a healthy population. Such test values are said to fall “within the normal range.” “Clinical remission” does not imply cessation of treatment. As used herein, “clinical remission” does not require that all clinical measures for a disease fall within the normal range. For example, a cat for which the serum fructosamine falls within the normal range, but for which the fasting serum glucose is greater than the upper limit of the normal range, is said to be in clinical remission.

As used herein, “normal ranges” can be dependent on the equipment and procedures of the testing laboratory and hence may vary from laboratory to laboratory. When values fall “within the normal range” as used herein, the phrase means within the range established by the particular laboratory providing the results at the time of measurement.

As used herein, “treatment-free remission” describes a state of remission that persists after cessation of administration of the therapeutic agent.

As used herein, “hyperglycemia-associated clinical signs” describes one or more of the characteristic signs of polydipsia, polyphagia, polyuria, or weight loss. Clinical signs of weight loss can be quantitated by direct measurement of the weight of a cat during an office visit. Recording of the other signs typically requires owner observation of cat behaviors or their consequences.

As used herein, the word “cat” when used as an adjective and the word “feline” are used interchangeably and mean of or pertaining to an animal from the family Felidae, including particularly a member of that family that is maintained as a pet or companion animal and that typically belongs to the genus Felis, species Silvestris catus or species catus and is often referred to as a domestic cat or house cat.

As used herein, the word cat when used as a noun refers to a feline animal.

As used herein, “managing diabetes” or “management of diabetes” refers to the process by which an owner or other person responsible for the care of an animal addresses the disease by specific measures intended to palliate or cure the disease or provide relief of symptoms or change the perceived health of the animal by various means. Such means can include a change in the diet of the animal, including provision of a special or prescription diet or other change in the type or amount of food offered, or the encouragement or provision of activities that result in increased exertion or metabolic energy expenditure, or the provision of herbal preparations, dietary supplements or medicaments.

As used herein, “blood glucose”, “blood glucose concentration” or “blood glucose levels” refer to measurements of glucose in whole blood. Typically, the sample drawn is capillary blood and the glucose is measured by a point-of-care device such as a glucometer.

As used herein, a “blood glucose curve” refers to the results of determining the glucose concentration in serial samples of whole blood obtained over a period of time typically ranging from 8 to 24 hours and undertaken as a means of assessing the degree of disease control and the appropriateness of the management, for example to determine if the amount of a therapeutic medicament is too great and an adversely low blood glucose is observed.

As used herein, “serum glucose”, “serum glucose concentration” or “serum glucose level” refers to a glucose concentration that is measured in the liquid phase of whole, typically venous, blood that has been allowed to coagulate. Serum glucose concentrations are often determined in clinical practices by automated procedures performed in a diagnostic testing laboratory.

As used herein, “serum fructosamine”, “serum fructosamine concentration” or “serum fructosamine level” refers to a fructosamine concentration that is measured in the liquid phase of whole, typically venous, blood that has been allowed to coagulate. Serum fructosamine concentrations are often determined in clinical practices by automated procedures performed in a diagnostic testing laboratory.

As used herein, “plasma glucose” or “plasma glucose concentration” refers to a glucose concentration that is obtained by measurement of the liquid phase of whole, typically venous, blood that has been separated from the cellular components of the blood in such a manner that the blood does not coagulate.

As used herein, the word “fasting” when applied to the circumstances surrounding the collection of a specimen for testing indicates that the animal from which the specimen was drawn had been deprived of food for an extended period of time, typically 6 hours or longer and not unusually overnight if the specimen is taken in the morning. A fasting sample is useful for the measurements of analytes such as glucose or lipids that are greatly affected by feeding.

As used herein, “reduction of hyperglycemia-associated clinical signs” as well as “an improvement of associated clinical signs” as it relates to hyperglycemia, means an improvement from the time of initiation of management of one or more of the signs of polydipsia, polyphagia, polyuria or the prevention of weight loss.

As used herein, an improvement in polydipsia means a decrease in the observed frequency or volume of water or fluids consumed, or a decrease in the frequency of seeking out unusual sources of water that the cat does not regularly consume.

As used herein, an improvement in polyphagia means a decrease in the amount of food consumed, or the frequency of begging or soliciting abnormal amounts of food, or the begging or soliciting of food under unusual circumstances, such as immediately following a feeding.

As used herein, an improvement in polyuria means a decrease in the frequency of urination or amount of urine produced, or a decrease in unusual behavior related to urination, including urination outside of a litter box provided for the purpose or flooding of the box.

As used herein, “prevention of weight loss” or “reducing the weight loss” means resulting in a decrease in body weight no greater than 1, 2, 3, 4, 5, 6, or 7% from the time of initiation of management. For avoidance of doubt “prevention of weight loss” or “reducing the weight loss” encompasses any increase in body weight from the time of initiation of management.

As used herein, the definition of “hypoglycemia” refers to the clinical state in which the measured blood glucose concentration falls below the upper limit of the range for the ISFM definition (Sparkes et al., 2015; J Feline Med Surg 17:235) of a blood glucose <3.0-3.5 mM (53-63 mg dL⁻¹). For avoidance of doubt, hypoglycemia means a blood glucose <63 mg dL⁻¹.

Clinical markers for diabetes include, but are not limited to, serum fructosamine levels, blood or serum glucose levels, or glycated hemoglobin levels. A management regimen can be for at least 1, 3, 7, 14, 28, or more days; or 1, 2, 3, 4, or more months or for the remainder of the lifespan. In some embodiments, the management regimen is 2 months. In some embodiments the clinical remission is permanent, that is, persists for the lifespan of the cat. In some embodiments a management-free remission is achieved. In some embodiments the management-free remission duration is at least 1, 3, 7, 14, 28, or more days; or 1, 2, 3, 4, or more months or for the remainder of the lifespan. The amount of time the management-free remission lasts will depend on a number of factors including the feline, its diet, and amount of daily exercise. As a non-limiting example, clinical remission can be identified by a serum fructosamine level at or below the upper limit of normal for the testing laboratory reference range. As an additional non-limiting example, clinical remission can be identified by a fasting plasma glucose level of at or below 170 mg dL⁻¹.

As used herein “upper limit of normal” or “ULN” of a testing laboratory reference range means the least upper bound of the range of values for a laboratory test that are thought to be found within the normal variation of specimens drawn from a healthy population. The upper limit of normal is typically provided by the testing laboratory in connection with the transmission of test results to the practitioner and may vary from laboratory to laboratory or from time to time within a laboratory, depending on the test calibration, test conduct, or specimen preparation.

The term “body condition score” (BCS) means a method for body composition analysis based upon an animal's body size and shape. As used herein BCS is determined using the methods disclosed in U.S. Pat. No. 6,691,639.

The term “fat” as applied to a feline means any feline that is determined to have an excess amount of body adipose tissue or a feline that is prone to developing an excess amount of body adipose tissue using techniques and methods known to health care providers and other skilled artisans. A feline is prone to becoming overweight if the feline has an inclination or a higher likelihood of developing excess adipose tissue when compared to an average feline in the general population. Generally, without limiting the definition, a feline is considered overweight if the feline's weight is 15% or more than its “ideal” body weight as defined by health care professionals or related skilled artisans, or a feline has a body condition score of more than 5 as determined by skilled artisans using the methods disclosed in U.S. Pat. No. 6,691,639.

The term “lean” as applied to a feline means any feline that is determined not to be overweight using techniques and methods known to health care providers and other skilled artisans. Generally, the feline's weight is less than 15% more than its “ideal” body weight as defined by health care professionals or related skilled artisans, or (4) a feline has a body condition score of less than 5 as determined by skilled artisans using the methods disclosed in U.S. Pat. No. 6,691,639.

As used herein “antidiabetic agent” refers to a composition comprising medicines, medications, or medicaments generally used in the treatment of diabetes in humans or the management of diabetes in animals. Common antidiabetic agents for the treatment of human T2DM include, but are not limited to alpha-glucosidase inhibitors, amylin analogs, biguanides, dipeptidyl peptidase 4 inhibitors, incretins or incretin mimetics, insulins, meglitinides, non-sulfonylurea secretagogues, SGLT2 inhibitors, sulfonylureas, and thiazolidinediones. It is generally accepted that oral medications for the treatment of T2DM in humans have little utility for the management of feline diabetes. To date no oral medication has been approved for the management of feline diabetes by a regulatory authority of the United States, the European Union, or Japan.

As used herein, “low carbohydrate diet” refers to the food intake of a feline from the time of initiation of management. In particular, a low carbohydrate diet is one where the relative amount of carbohydrates consumed does not exceed a certain threshold level. Low carbohydrate diets typically include less than 40%, 35%, 30%, 26%, 20%, 15%, 12% or lower percentage of calories from carbohydrate.

As used herein, “diabetic diet” refers to the food intake of a feline from the time of initiation of management. In particular, a diabetic diet is one that includes relatively high amounts of protein and low amounts of carbohydrates. High amounts of protein include 60%, 65%, 70%, 75%, 80% or greater percentage of calories from protein, while low amounts of carbohydrate are as defined above. In particular embodiments, a diabetic diet does not include dry cat food.

As used herein, an “elevated frequency of diarrhea or loose stools” means any frequency of diarrhea or loose stools that exceeds that of the unmedicated animal by more than ten percent of defecations. Diarrhea, as used herein, does not include incidental causes such as infection by a bacteria, virus, coccidian, or intestinal worms, but instead refers to diarrhea resulting from SGLT1 inhibition. In some embodiments, “diarrhea” refers to loose or liquid stools occurring at least once a day for at least three days during the course of treatment.

As used herein “SGLT inhibitor” refers to compounds that have activity against both SGLT1 and SGLT2, and especially those compounds that have a favorable proportion of SGLT2 activity to SGLT1 activity, such that the benefit of glucosuria and impaired absorption of enteric carbohydrate is accompanied by a low risk of unfavorable gastrointestinal symptoms such as diarrhea, loose stools, flatulence and bloating.

As used herein, the term “administering” means delivering by oral, buccal, nasal, rectal, vaginal or cutaneous routes or other topical contact, or by intravenous, intraperitoneal, intramuscular, intralesional or subcutaneous routes, or by the implantation of a slow-release device or preparation such as a pump, gel, reservoir or erodible substance to a subject. Administration can be by any route including parenteral, and transmucosal (e.g., oral, nasal, vaginal, rectal, or transdermal). Parenteral administration includes, e.g., intravenous, intramuscular, intra-arterial, intradermal, subcutaneous, intraperitoneal, intraventricular, intrathecal and intracranial. Other modes of delivery include, but are not limited to, the use of liposomal formulations, intravenous infusion, transdermal patches, and the like.

As used herein, a method of managing, inducing, reducing, improving, or preventing a disorder, disease, or condition comprising administering a compound or composition may also mean the use of a compound or composition for managing, inducing, reducing, improving, or preventing a disorder, disease, or condition, as well as the use of a compound or composition for preparation of a medicament for managing, inducing, reducing, improving, or preventing a disorder, disease, or condition.

### II. Methods of Use

In some aspects, provided herein are methods of reducing weight loss in a feline in need thereof, which methods include administering to the feline in need thereof a total daily dosage of about 2 to 50 mg of Compound 1, having the formula:

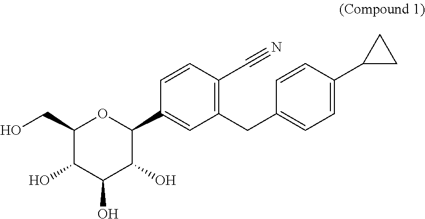 [SMILES: N#Cc1ccc([C@@H]2O[C@H](CO)[C@@H](O)[C@H](O)[C@H]2O)cc1Cc1ccc(C2CC2)cc1]

or a pharmaceutically acceptable form thereof.

The reduced weight loss observed will vary depending on a variety of factors including the age and breed of the feline. In some embodiments, the weight loss of the feline in need of a reduction thereof is no more than a 7% decrease in the body weight of the feline as compared to the body weight of the feline before initiation of treatment. In some embodiments, the weight loss of the feline in need of a reduction thereof is no more than a 6% decrease in the body weight of the feline as compared to the body weight of the feline before initiation of treatment. In some embodiments, the weight loss of the feline in need of a reduction thereof is no more than a 5% decrease in the body weight of the feline as compared to the body weight of the feline before initiation of treatment. In some embodiments, the weight loss of the feline in need of a reduction thereof is no more than a 4% decrease in the body weight of the feline as compared to the body weight of the feline before initiation of treatment. In some embodiments, the weight loss of the feline in need of a reduction thereof is no more than a 3% decrease in the body weight of the feline as compared to the body weight of the feline before initiation of treatment. In some embodiments, the weight loss of the feline in need of a reduction thereof is no more than a 2% decrease in the body weight of the feline as compared to the body weight of the feline before initiation of treatment. In some embodiments, the weight loss of the feline in need of a reduction thereof is no more than a 1% decrease in the body weight of the feline as compared to the body weight of the feline before initiation of treatment.

In some aspects, provided herein are methods of increasing the weight of a feline in need thereof, said method comprising administering to the feline in need thereof of a total daily dosage of about 2 to 50 mg of Compound 1. Measurements to determine an increase in the weight of the feline are done by comparing the body weight after (or during) treatment to the body weight of the feline before initiation of treatment.

In some embodiments, the body weight of the feline increases about 1, 2, 3, 4, 5% or more as compared to the body weight of the feline before initiation of treatment. In some embodiments, the body weight of the feline increases about 1% as compared to the body weight of the feline before initiation of treatment. In some embodiments, the body weight of the feline increases about 2% as compared to the body weight of the feline before initiation of treatment. In some embodiments, the body weight of the feline increases about 3% as compared to the body weight of the feline before initiation of treatment. In some embodiments, the body weight of the feline increases about 4% as compared to the body weight of the feline before initiation of treatment. In some embodiments, the body weight of the feline increases about 5% as compared to the body weight of the feline before initiation of treatment.

Methods of reducing weight loss or increasing the weight of a feline can help stabilize or reverse a recent weight change in a feline. These methods are useful for both diabetic and non-diabetic felines. Thus, in some embodiments, the feline in need thereof is a diabetic feline. In some embodiments, the feline in need thereof is a non-diabetic feline.

In some aspects, provided herein are methods for managing the diabetes of a feline exhibiting an IGF-1 concentration greater than the upper limit of normal for the testing laboratory reference range, comprising administering to the feline in need thereof a total daily dosage of about 2 to 50 mg of Compound 1. In some embodiments, the upper limit of normal for the testing laboratory reference range is 92 nmol/L.

Felines with all types of body compositions are suitable for the methods described herein. This includes fat felines, lean felines, and felines with standard body composition. In some embodiments, the felines in the methods described herein are fat felines. In some embodiments, fat felines are felines that are 15% of more than the “ideal” body weight as defined by health care professionals. Often health care professions define ideal body weight based on the breed and age of the feline. In some embodiments, fat felines are felines that have a body condition score of more than 5. In some embodiments, the felines in the methods described herein are lean felines. In some embodiments, lean felines are felines that are 15% of less than the “ideal” body weight as defined by health care professionals. Often health care professions define ideal body weight based on the breed and age of the feline. In some embodiments, lean felines are felines that have a body condition score of less than 5.

The body condition score (BCS) is a scoring system that incorporates various assessments of a feline to report on the body condition of the feline. Further details on the assessments undertaken are described in U.S. Pat. No. 6,691,639, the contents of which are incorporated by reference herein for all purposes. In short, the BCS provides a scoring system from 1 to 9 which reports on the body condition of the feline, where 1 is emaciated and 9 is grossly obese. In some embodiments, the felines in the methods described herein have a body condition score (BCS) of 1, meaning the feline is emaciated. In some embodiments, the felines in the methods described herein have a BCS of 2, meaning the feline is very thin. In some embodiments, the felines in the methods described herein have a BCS of 3, meaning the feline is thin. In some embodiments, the felines in the methods described herein have a BCS of 4, meaning the feline is underweight. In some embodiments, the felines in the methods described herein have a BCS of 5, meaning the feline has an ideal weight. In some embodiments, the felines in the methods described herein have a BCS of 6, meaning the feline is overweight. In some embodiments, the felines in the methods described herein have a BCS of 7, meaning the feline is heavy. In some embodiments, the felines in the methods described herein have a BCS of 8, meaning the feline is obese. In some embodiments, the felines in the methods described herein have a BCS of 9, meaning the feline is grossly obese.

In some embodiments, the methods provided herein include administering to a feline in need thereof a low carbohydrate diet and a total daily dosage comprising about 2 to 50 mg of Compound 1. Carbohydrates are commonly present in commercial cat foods, and maintaining a low carbohydrate diet will improve the clinical pathology of the feline. In some embodiments, said low carbohydrate diet is a canned diet. Felines on a canned diet will not be fed any dry cat food. In some embodiments, said low carbohydrate diet is a diabetic diet. A diabetic diet is one that is generally high in protein and is low in carbohydrates. In some embodiments, a diabetic diet includes little or no dry cat food. In some embodiments, said low carbohydrate diet is a ketogenic diet. Ketogenic diets include diets that are high in seafood, meat, poultry, and eggs. In some embodiments, said low carbohydrate diet is a grain-free diet.

In some embodiments, said low carbohydrate diet contains less than 40% of calories in the form of carbohydrates. In some embodiments, said low carbohydrate diet contains less than 35% of calories in the form of carbohydrates. In some embodiments, said low carbohydrate diet contains less than 30% of calories in the form of carbohydrates. In some embodiments, said low carbohydrate diet contains less than 26% of calories in the form of carbohydrates. In some embodiments, said low carbohydrate diet contains less than 12% of calories in the form of carbohydrates.

In some embodiments, Compound 1 is a crystalline form of 2-(4-cyclopropylbenzyl)-4-((2S,3R,4R,5S,6R)-3,4,5-trihydroxy-6-(hydroxymethyl)tetrahydro-2H-pyran-2-yl)benzonitrile, having the formula

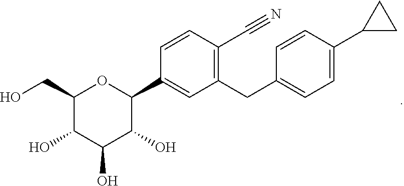 [SMILES: N#Cc1ccc([C@@H]2O[C@H](CO)[C@@H](O)[C@H](O)[C@H]2O)cc1Cc1ccc(C2CC2)cc1]

In some embodiments, the therapeutically effective amount of Compound 1 is a total daily dosage of about 2 mg to 50 mg (e.g., about 2, 3, 4, 5, 7.5, 10, 12.5, 15, 17.5, 20, 22.5, 25, 35, 40, 45 or 50 mg day⁻¹). In some embodiments, the total daily dosage of Compound 1 is about 10 to 20 mg. In some embodiments, the total daily dosage of Compound 1 is about 15 mg.

In some embodiments, the total daily dosage of Compound 1 is dependent on the weight of the feline. Thus, in some embodiments, the total daily dosage of Compound 1 is about 0.1 to 10 mg kg⁻¹. In some embodiments, the total daily dosage of Compound 1 is about 0.3 to 3 mg kg′. In some embodiments, the total daily dosage of Compound 1 is about 0.5 to 2 mg kg′. In some embodiments, the total daily dosage of Compound 1 is about 1 mg kg′.

Compound 1 may be administered to felines via a number of suitable routes. In some embodiments, Compound 1 is administered orally. Further methods of administration are discussed in the sections below.

In some embodiments, Compound 1 is administered in combination with an additional therapeutic agent. In some embodiments, Compound 1 is administered as a monotherapy.

Advantageously, administration of Compound 1 does not need to be timed with a meal or other event. In some embodiments, the total daily dosage is administered once daily independent of other activities (including meal timing). In some embodiments, the total daily dosage is administered twice daily independent of other activities (including meal timing). In some embodiments, the dosage is admixed with the cat's food. In some embodiments the dosage is delivered to the cat as a single solid dosage form. In some embodiments the dosage is delivered as an oral solution or oral suspension. In some embodiments the maximum fluid volume delivered is 1 mL. In some embodiments the maximum volume delivered is 0.5 mL. In some embodiments the dosage is adjusted according to the weight of the cat. In some embodiments a single dosage strength is provided for all cats.

Over the course of management, the serum fructosamine levels and/or blood or serum glucose levels can be monitored for evidence of glycemic control. Clinical signs such as polyuria, polydipsia, polyphagia or weight loss may also be monitored. If continuing signs of diabetes persist, the management plan may be changed to include other features, including other medications.

When a feline is maintaining clinical remission, the feline sustains the positive therapeutic benefit received from the therapeutic regimen received. In some embodiments, clinical remission is maintained when the feline does not present one or more clinical markers for feline diabetes. As discussed above, the symptoms of feline diabetes include elevated levels of serum fructosamine, elevated levels of blood or serum glucose levels, polyuria, polydipsia, and polyphagia.

In some embodiments, a feline maintaining clinical remission is determined by the feline's serum fructosamine levels. In some embodiments, the feline's serum fructosamine level is compared to the upper limit of normal for the testing laboratory reference range. In some embodiments, the upper limit of normal for the testing laboratory reference is about 356 μmol L⁻¹ or about 275 μmol L⁻¹. In some embodiments, the feline in clinical remission exhibits a serum fructosamine level at or below the upper limit of normal for the testing laboratory reference range. In some embodiments, the feline in clinical remission exhibits a serum fructosamine level at or below 360 μmol L⁻¹. In some embodiments, the feline in clinical remission exhibits a serum fructosamine level at or below 350 μmol L⁻¹. In some embodiments the feline in clinical remission exhibits a serum fructosamine level that is at or below the upper limit of normal for the testing laboratory to which the specimen has been submitted.

In some embodiments, a feline maintaining clinical remission is determined by the feline's blood or serum glucose levels. In some embodiments, the feline in clinical remission maintains a blood or serum glucose level of less than 250 mg dL⁻¹. In some embodiments, the feline in clinical remission maintains a blood or serum glucose level of less than 200 mg dL⁻¹. In some embodiments, the feline in clinical remission maintains a blood or serum glucose level of less than 190 mg dL⁻¹. In some embodiments, the feline in clinical remission maintains a blood or serum glucose level of less than 180 mg dL⁻¹. In some embodiments, the feline in clinical remission maintains a blood or serum glucose level of less than 170 mg dL⁻¹. In some embodiments, the feline in clinical remission maintains a blood or serum glucose level of less than 160 mg dL⁻¹. In some embodiments, the feline in clinical remission maintains a blood or serum glucose level of less than 150 mg dL⁻¹.

The methods described herein reduce, lessen, or eliminate symptoms of feline diabetes. For example, in some embodiments, a feline's serum fructosamine level, as measured after completion of a management regimen, is reduced in said feline. In some embodiments, a feline's blood or serum glucose level, as measured after completion of a management regimen, is reduced in said feline.

In some embodiments, the feline's serum fructosamine level is reduced by at least about 20% after completion of said management regimen. In some embodiments, the feline's serum fructosamine level is reduced by at least about 30% after completion of said management regimen. In some embodiments, the feline's serum fructosamine level is reduced by at least about 40% after completion of said management regimen. In some embodiments, the feline's serum fructosamine level is reduced by at least about 50% after completion of said management regimen.

In some embodiments, the feline's serum fructosamine level is less than the upper limit of normal for the testing laboratory reference range after completion of said management regimen. In some embodiments, the upper limit of normal for the testing laboratory reference is about 356 μmol or about 275 μmol L⁻¹. In some embodiments, the feline's serum fructosamine level is less than 500 μmol after completion of said management regimen. In some embodiments, the feline's serum fructosamine level is less than 450 μmol L⁻¹ after completion of said management regimen. In some embodiments, the feline's serum fructosamine level is less than 400 μmol L⁻¹ after completion of said management regimen. In some embodiments, the feline's serum fructosamine level is less than 350 μmol L⁻¹ after completion of said management regimen.

In some embodiments, the feline's blood or serum glucose level is reduced by at least about 20% after completion of said management regimen. In some embodiments, the feline's blood or serum glucose level is reduced by at least about 30% after completion of said management regimen. In some embodiments, the feline's blood or serum glucose level is reduced by at least about 40% after completion of said management regimen. In some embodiments, the feline's blood or serum glucose level is reduced by at least about 50% after completion of said management regimen.

In some embodiments, the feline's blood or serum glucose level is less than 250 mg dL⁻¹ after completion of said management regimen. In some embodiments, the feline's blood or serum glucose level is less than 200 mg dL⁻¹ after completion of said management regimen. In some embodiments, the feline's blood or serum glucose level is less than 190 mg dL⁻¹ after completion of said management regimen. In some embodiments, the feline's blood or serum glucose level is less than 180 mg dL⁻¹ after completion of said management regimen. In some embodiments, the feline's blood or serum glucose level is less than 170 mg dL⁻¹ after completion of said management regimen. In some embodiments, the feline's blood or serum glucose level is less than 160 mg dL⁻¹ after completion of said management regimen. In some embodiments, the feline's blood or serum glucose level is less than 150 mg dL⁻¹ after completion of said management regimen.

As discussed supra, it is favorable to have an activity on SGLT1 that is lower than the activity of SGLT2, and that SGLT1 inhibition can provoke diarrhea. Thus, SGLT inhibitors of the present disclosure provide a pharmacodynamic effect in the treatment of feline diabetes at a dosage level beneath the threshold level of adverse enteric effects. As such, in an additional aspect, provided herein are methods of managing feline diabetes, which includes administering to a feline in need thereof an effective amount of a SGLT inhibitor, wherein said effective amount is no more than 10 to 30% of the dose required to produce an elevated frequency of diarrhea or loose stool in a healthy feline. In some embodiments, the healthy feline is on a commercial dry food diet. In some embodiments, the healthy feline is one which is not exhibiting elevated frequency of diarrhea or loose stool prior to administration of a SGLT inhibitor. In some embodiments, the healthy feline is non-diabetic.

In some embodiments, the effective amount is no more than 10, 12, 16, 18, 20, 22, 24, 26, 28, or 30% of the dose required to produce an elevated diarrhea or loose stool in a healthy feline. In some embodiments, the effective amount is no more than 30% of the dose required to produce an elevated diarrhea or loose stool in a healthy feline. In some embodiments, the effective amount is no more than 20% of the dose required to produce an elevated diarrhea or loose stool in a healthy feline. In some embodiments, the effective amount is no more than 10% of the dose required to produce an elevated diarrhea or loose stool in a healthy feline.

An effective amount includes a dose that produces at least 90% percent of the maximum pharmacodynamics effect of said SGLT inhibitor.

Also provided herein are methods of managing feline diabetes, which includes administering to a feline in need thereof an effective amount of a SGLT inhibitor, wherein said SGLT inhibitor produces an elevated frequency of diarrhea or loose stools in a healthy feline at a dose of no less than 3 to 10 times said effective amount. In some embodiments, the healthy feline is on a commercial dry food diet. In some embodiments, the healthy feline is one which is not exhibiting elevated frequency of diarrhea or loose stool prior to treatment. In some embodiments, the healthy feline is non-diabetic.

In some embodiments, the SGLT inhibitor produces an elevated frequency of diarrhea or loose stools in a healthy feline at a dose of no less than 3, 4, 5, 6, 7, 8, 9, or 10 times the effective amount. In some embodiments, the SGLT inhibitor produces an elevated frequency of diarrhea or loose stools in a healthy feline at a dose of no less than 3 times the effective amount. In some embodiments, the SGLT inhibitor produces an elevated frequency of diarrhea or loose stools in a healthy feline at a dose of no less than 5 times the effective amount. In some embodiments, the SGLT inhibitor produces an elevated frequency of diarrhea or loose stools in a healthy feline at a dose of no less than 10 times the effective amount.

An effective amount includes a dose that produces at least 90% percent of the maximum pharmacodynamics effect of said SGLT inhibitor.

The methods described herein are useful in managing all forms of feline diabetes. In some embodiments, the feline diabetes is type 1 diabetes. In some embodiments, the feline diabetes is type 2 diabetes.

### III. Pharmaceutical Compositions

Compound 1 can be prepared in various compositions suitable for delivery to a subject. A composition suitable for administration to a subject typically comprises Compound 1, (or a pharmaceutically acceptable form thereof and a pharmaceutically acceptable carrier.

Compound 1 can be incorporated into a variety of formulations for therapeutic administration. More particularly, Compound 1 can be formulated into pharmaceutical compositions, together or separately, by formulation with appropriate pharmaceutically acceptable carriers or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of a compound of the present invention can be achieved in various ways, including oral, buccal, parenteral, intravenous, intradermal (e.g., subcutaneous, intramuscular), transdermal, etc., administration. Moreover, Compound 1 can be administered in a local rather than systemic manner, for example, in a depot or sustained release formulation.

The pharmaceutical compositions for the administration of Compound 1 can conveniently be presented in unit dosage form and can be prepared by any of the methods known in the art of pharmacy and drug delivery. All methods include the step of bringing the active ingredient into association with a carrier containing one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation.

Suitable formulations for use in the present invention are found in Remington: THE SCIENCE AND PRACTICE OF PHARMACY, 21st Ed., Gennaro, Ed., Lippincott Williams & Wilkins (2003), which is hereby incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

In some embodiments, Compound 1 is prepared for delivery in a sustained-release, controlled release, extended-release, timed-release or delayed-release formulation, for example, in semipermeable matrices of solid hydrophobic polymers containing the therapeutic agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients (see, for example, Huang, et al. Drug Dev. Ind. Pharm. 29:79 (2003); Pearnchob, et al. Drug Dev. Ind. Pharm. 29:925 (2003); Maggi, et al. Eur. J. Pharm. Biopharm. 55:99 (2003); Khanvilkar, et al., Drug Dev. Ind. Pharm. 228:601 (2002); and Schmidt, et al., Int. J. Pharm. 216:9 (2001)). Sustained-release delivery systems can, depending on their design, release the compounds over the course of hours or days, for instance, over 4, 6, 8, 10, 12, 16, 20, 24 hours or more. Usually, sustained release formulations can be prepared using naturally-occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

The sustained or extended-release formulations can also be prepared using natural ingredients, such as minerals, including titanium dioxide, silicon dioxide, zinc oxide, and clay (see, U.S. Pat. No. 6,638,521, herein incorporated by reference). Exemplified extended release formulations that can be used in delivering Compound 1 (in any of the forms described herein) include those described in U.S. Pat. Nos. 6,635,680; 6,624,200; 6,613,361; 6,613,358, 6,596,308; 6,589,563; 6,562,375; 6,548,084; 6,541,020; 6,537,579; 6,528,080 and 6,524,621, each of which is hereby incorporated herein by reference. Controlled release formulations of particular interest include those described in U.S. Pat. Nos. 6,607,751; 6,599,529; 6,569,463; 6,565,883; 6,482,440; 6,403,597; 6,319,919; 6,150,354; 6,080,736; 5,672,356; 5,472,704; 5,312,817 and 5,296,483, each of which is hereby incorporated herein by reference. Those skilled in the art will readily recognize other applicable sustained release formulations.

For oral administration, Compound 1 can be readily formulated by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient in need thereof. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

Tablets of the current disclosure contain the active ingredient in admixture with non-toxic pharmaceutically acceptable excipients which are suitable for the manufacture of tablets. These excipients may be, for example, inert diluents, such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated, enterically or otherwise, by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for controlled release.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

Formulations for oral use may also be presented as hard gelatin capsules wherein the active ingredient is mixed with an inert solid diluent, for example, microcrystalline cellulose, lactose, starch, pregelatinized starch or kaolin, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono-diglycerides, PEG esters and the like.

In some instances, Compound 1 can be formulated for parenteral administration by injection, e.g., by bolus injection or continuous infusion. For injection, the compound can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, Compound 1 can be formulated in aqueous solutions, preferably in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain agents such as suspending, stabilizing and/or dispersing agents.

Pharmaceutical formulations for parenteral administration include aqueous solutions of Compound 1 (in any of the forms noted herein) in water-soluble form. Additionally, suspensions of Compound 1 can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions. Alternatively, Compound 1 can be in powder form for constitution with a suitable vehicle, e.g., sterile pyrogen-free water, before use. Methods of formulating SGLT2 inhibitors are known in the art and are described, for example, in US2017/0056366, the contents of which is herein incorporated by reference for all purposes.

Systemic administration can also be by transmucosal or transdermal means. For transmucosal or transdermal administration, penetrants appropriate to the barrier to be permeated are used in the formulation. For topical administration, Compound 1 can be formulated into ointments, creams, salves, powders and gels. In one embodiment, the transdermal delivery agent can be DMSO. Transdermal delivery systems can include, e.g., patches. For transmucosal administration, penetrants appropriate to the barrier to be permeated are used in the formulation. Such penetrants are generally known in the art. Exemplified transdermal delivery formulations that can find use in the present invention include those described in U.S. Pat. Nos. 6,589,549; 6,544,548; 6,517,864; 6,512,010; 6,465,006; 6,379,696; 6,312,717 and 6,310,177, each of which are hereby incorporated herein by reference.

In addition to the formulations described previously, Compound 1 can also be formulated as a depot preparation. Such long acting formulations can be administered by implantation (for example subcutaneously or intramuscularly) or by intramuscular injection. Thus, for example, Compound 1 can be formulated with suitable polymeric or hydrophobic materials (for example as an emulsion in an acceptable oil) or ion exchange resins, or as sparingly soluble derivatives, for example, as a sparingly soluble complex or salt.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

### IV. Pharmaceutical Dosage Forms

The present disclosure includes pharmaceutical dosage forms of Compound 1, or a pharmaceutically acceptable form thereof. The dosage forms described herein are suitable for oral administration to a subject. The dosage form may be in any form suitable for oral administration, including, but not limited to, a capsule or a tablet.

In some embodiments, the present disclosure provides an oral liquid dosage form permitting the delivery of a pharmaceutically acceptable dosage containing 2 to 50 mg of Compound 1, (velagliflozin), having the formula:

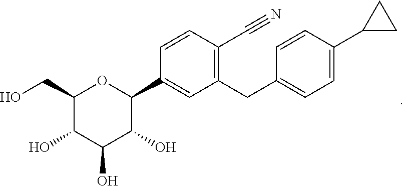 [SMILES: N#Cc1ccc([C@@H]2O[C@H](CO)[C@@H](O)[C@H](O)[C@H]2O)cc1Cc1ccc(C2CC2)cc1]

In some embodiments, the amount of Compound 1 is from about 0.1 to 10 mg/kg. In some embodiments, the amount of Compound 1 is from about 0.3 to 3 mg/kg. In some embodiments, the amount of Compound 1 is about 1 mg/kg.

In some embodiments, a single unit dosage form of Compound 1 is provided in the form of a capsule. In some embodiments, a single unit dosage form of Compound 1 is provided in the form of a tablet.

In some embodiments, the single unit dosage form is in a capsule of size #0, #1, #2, #3, #4, or #5. In some embodiments, the single unit dosage form is in a capsule of size #4. In some embodiments, the single unit dosage form is in a capsule of size #5.

### V. Kits

Also provided herein are kits comprising pharmaceutical compositions and dosage forms of Compound 1, or forms thereof.

In some aspects, the present invention provides a kit that includes Compound 1. Some of the kits described herein include a label describing a method of administering Compound 1.

Some of the kits described herein include a label describing a method of managing feline diabetes. In some embodiments, the kits described herein include a label describing a method of reducing a feline's serum fructosamine and/or blood or serum glucose levels.

The compositions of the present invention, including but not limited to, compositions comprising Compound 1 in a bottle, jar, vial, ampoule, tube, or other container-closure system approved by the United States Food and Drug Administration (FDA) or other regulatory body, which may provide one or more dosages containing the compounds. The package or dispenser may also be accompanied by a notice associated with the container in a form prescribed by a governmental agency regulating the manufacture, use, or sale of pharmaceuticals, the notice indicating approval by the agency. In certain aspects, the kit may include a formulation or composition as described herein, a container closure system including the formulation or a dosage unit form including the formulation, and a notice or instructions describing a method of use as described herein.

### EXAMPLES

The following examples are offered for illustrative purposes, and are not intended to limit the invention in any manner. Those of skill in the art will readily recognize a variety of noncritical parameters which can be changed or modified to yield essentially the same results.

### Example 1. Effects of Velagliflozin in Nondiabetic Cats

Velagliflozin (2-(4-cyclopropylbenzyl)-4-((2S,3R,4R,5S,6R)-3,4,5-trihydroxy-6-(hydroxymethyl)tetrahydro-2H-pyran-2-yl)benzonitrile, CAS number 946525-65-1; Compound 1) can be prepared by a variety of means, including as described in US2015/0164856A1 and is commercially available, for example as catalog number HY-109018 from MedChemExpress. Velagliflozin formulated in gelatin capsules was administered to healthy purpose-bred cats once a day for three days and the urinary glucose excretion recorded over the three consecutive 24-hour periods following dosing. Velagliflozin was administered at three dose levels: 1 mg, 3 mg and 10 mg per cat per day. The mean daily glucose excretion over three days was 2527 mg, 3601 mg and 5256 mg for mean dosages of 0.318 mg/kg, 0.953 mg/kg and 3.071 mg/kg, respectively.

### Example 2. Effects of Velagliflozin in Diabetic Cats

In a field effectiveness study, client-owned cats diagnosed with diabetes mellitus based on: i) two separate fasting (>6 h) blood glucose measurements >250 mg dL⁻¹; ii) glucosuria; iii) fructosamine >25% above the upper limit of normal for the testing laboratory, and iv) one or more of the following: polyuria/polydipsia, polyphagia and/or weight loss (documented in the cat's medical records) can be enrolled. Cats with suspected diabetes mellitus can be evaluated during visit 1 (within 7 days prior to day 0). Eligible cats can be enrolled during visit 2 (day 0) and management can be initiated using velagliflozin administered orally once daily. Cats can be prescribed a low-carbohydrate diet. Cats would return to the clinic for evaluation of glycemic control during visits 3 (day 14±3), 4 (day 28±3), and 5 (day 56±3). The treatment period can be from visit 2 (day 0) through visit 5 (day 56±3). A cat can be brought back to the clinic at any time for unscheduled visits if such visits are determined to be necessary by the owner or investigator. Eight-hour blood glucose curves (blood samples collected every 2 h±15 min for 8 h) can be conducted at every visit starting with visit 2 (day 0) and blood glucose can be measured using an AlphaTRAK 2 glucometer (Abbott Laboratories). Blood samples for hematology and serum chemistry can be collected at screening and during each scheduled visit after initiation of dosing. A central laboratory would be used to evaluate all clinical pathology samples (blood, serum, and urine) that are not analyzed in the clinic for blood glucose curves.

Cats exposed to velagliflozin in this study will be perceived by their owners to have shown improvement in polydipsia, polyphagia and polyuria. Of the three owner-evaluated signs, polyphagia will be the least likely to have been found to be improved. Weight gain and reduction in weight loss, despite the caloric wasting induced by velagliflozin, will be observed.

Responsibility for assessing clinical signs of diabetes mellitus is divided between owners and treating veterinarians. Weight is recorded at each visit by the veterinarian, and the signs of polydipsia, polyuria and polyphagia are recorded at each visit by the owner, using a four-point (0 to 3) integer score in which low scores represent favorable ratings.

In addition to providing a measure of the status of the cat as a function of time, the quantitative assessments can be used to produce a binary outcome of success or failure at study completion.

Cats will often be presented to a veterinarian because their owners have observed that they are losing weight, despite consuming more food than usual. However despite the glucosuria induced by velagliflozin, treatment with velagliflozin will increase the average weight of a cat or reduce the weight loss of a cat.

### Example 3. Effects of Velagliflozin in Diabetic Cats with Elevated IGF-1

Acromegaly-associated diabetes in cats represents a distinct etiology with particular challenges for management. Very high insulin doses are often required to overcome the profound insulin resistance typically encountered in such cases. In addition to the morphological changes that accompany disease of longstanding duration, elevated IGF-1 concentrations are pathognomonic for acromegaly in cats. Velagliflozin can be used to manage the diabetes of cats with diabetes mellitus and elevated IGF-1 concentrations.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of reducing the weight loss of a feline in need thereof, said method comprising administering to said feline a total daily dosage of about 2 to 50 mg of Compound 1, having the formula: 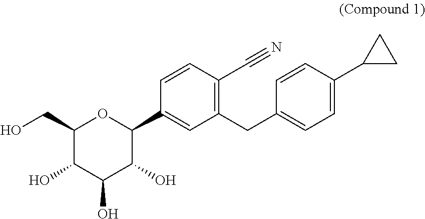 [SMILES: N#Cc1ccc([C@@H]2O[C@H](CO)[C@@H](O)[C@H](O)[C@H]2O)cc1Cc1ccc(C2CC2)cc1]
or a pharmaceutically acceptable form thereof.

2. The method of claim 1, wherein the weight loss of the feline in need of a reduction thereof is no more than a 5% decrease in the body weight of the feline as compared to the body weight of the feline before initiation of treatment.

3.-6. (canceled)

7. A method of increasing the weight of a feline in need thereof, said method comprising administering to said feline a total daily dosage of about 2 to 50 mg of Compound 1.

8. The method of claim 1, wherein the feline in need thereof is a diabetic feline.

9. (canceled)

10. A method for managing the diabetes of a feline exhibiting an IGF-1 concentration greater than the upper limit of normal for the testing laboratory reference range, comprising administering to said feline a total daily dosage of about 2 to 50 mg of Compound 1.

11. The method of claim 10, wherein the upper limit of normal for the testing laboratory reference range of IGF-1 is 92 nmol/L.

12.-13. (canceled)

14. The method of claim 1, wherein the feline in need thereof has a body condition score (BCS) of 1 or 2, meaning the feline is emaciated or very thin.

15. (canceled)

16. The method of claim 1, wherein the feline in need thereof has a body condition score (BCS) of 3 or 4, meaning the feline is thin or underweight.

17.-22. (canceled)

23. The method of claim 1, said method comprising administering to said feline a low carbohydrate diet and a total daily dosage comprising about 2 to 50 mg of Compound 1.

24.-27. (canceled)

28. The method of claim 23, wherein said low carbohydrate diet contains less than 40% of calories in the form of carbohydrates.

29.-39. (canceled)

40. The method of claim 10, wherein Compound 1 is administered in a management regimen, said management regimen lasting for a period of at least 28 days.

41. (canceled)

42. The method of claim 10, wherein Compound 1 is the only antidiabetic agent administered to said feline.

43. The method of claim 10, wherein administration of Compound 1 produces clinical remission in said feline.

44. The method of claim 40, wherein Compound 1 is administered to said feline having a serum fructosamine level greater than the upper limit of normal for the testing laboratory reference range prior to initiating management.

45. (canceled)

46. The method of claim 40, wherein Compound 1 is administered to said feline having a serum fructosamine level of 450 μmol/L or higher prior to initiating management.

47. The method of claim 40, wherein Compound 1 is administered to said feline having a blood or serum glucose level of 170 mg dL⁻¹ or higher prior to initiating management.

48.-55. (canceled)

56. The method of claim 40, wherein the serum fructosamine level of said feline is less than the upper limit of normal for the testing laboratory reference range after completion of said management regimen.

57.-58. (canceled)

59. The method of claim 40, wherein the blood or serum glucose level of said feline is reduced by at least about 20% after completion of said management regimen.

60.-70. (canceled)

71. A method of managing feline diabetes, said method comprising administering to a feline in need thereof an effective amount of a SGLT inhibitor,
wherein said effective amount is no more than 10 to 30% of the dose required to produce an elevated frequency of diarrhea or loose stool in a healthy feline.

72.-75. (canceled)

76. The method of claim 71, wherein said effective amount is a dose that produces about 90% the maximum pharmacodynamic effect of said SGLT inhibitor.
